(19) United States
(12) Patent Application Publication
(10) Pub. No.: US 2009/0081781 A1
(43) Pub. Date: Mar. 26, 2009

(54) Synthetic and standardized prion infectuous material, and uses thereof as an injecting inoculum

(75) Inventors: Jean-Thierry Aubin, Paris (FR); Bruno You, L'hay-Les-Roses (FR); Benoit Flan, Limours (FR)

Correspondence Address: BIRCH STEWART KOLASCH & BIRCH, PO BOX 747, FALLS CHURCH, VA, 22040-0747, US

(73) Assignee: Laboratoire Francais Du Fractionnement Et Des Biotechnologies, Les Ulis (FR)

(21) Appl. No.: 11/919,715

(22) PCT Filed: May 4, 2006
(86) PCT No.: PCT/FR2006/000999

§ 371 Date: Jun. 30, 2008

(30) Foreign Application Priority Data
May 4, 2005 (FR) 05 04545

(51) Int. Cl. C12N 5/10; C12N 5/08

(52) U.S. Cl. 435/354; 435/325; 435/352; 435/368

(57) ABSTRACT

The invention concerns a novel, synthetic, standardised, soluble, reproducible and easy-to-handle infectious material of the prion type, consisting of a cell lysate or culture supernatant from stable transgenic cells expressing a prion protein PrP and supporting replication of the pathogenic form, PrPsc, of the said Prp.

Synthetic and standardised prion infectious material, and uses thereof as an infecting inoculum.

The invention concerns a synthetic, standardised material of the prion type and its uses as an infecting inoculum.

### SCOPE OF THE INVENTION

The term transmissible spongiform encephalitis (TSE) describes a group of genetic or acquired diseases characterised by central nervous system (SNC) degeneration, known in humans as, amongst others Creutzfeld-Jacob disease (CJD) but which also affects other mammals such as sheep (Scrapie) and cattle (bovine spongiform encephalitis). The etiological agent of these diseases has properties placing it in a group known as “non-conventional transmissible agents” (NCTAS). The causative agent is not known, but the disease is typified by the presence of an extracellular protein known as prion protein (PrP). PrP changes in the course of the disease to an insoluble form which is at least partially resistant to proteases such as proteinase K, and which accumulates in cells causing their death. This abnormal and pathogenic form of PrP, known as PrPsc, ensues from a change in the conformation of protein prion PrP. No changes in the expression of the gene coding for PrP or modifications in its translation have been evidenced (P. Brown, Transfusion, 41, 4333-436, 2001 and D. Völkel et al, Transfusion, 41, 441-448, 2001).

In vitro or in vivo transgenesis contributes largely to our understanding of TSE. Thus, mice whose gene coding for PrPsc has been inactivated become highly resistant to the experimentally-induced disease. Conversely, mice and cell cultures expressing (or over-expressing) pathological transgenes cloned from the PrP gene of subjects with familial TSE or from xeno-genes, mimic the corresponding disease or are sensitive to an inoculum obtained from infected subjects belonging to the same species as the transgenes. Transgenic cell lines are also used as vitro models to test for molecules interacting with the trans-conformation of PrP to PrPsc.

On the basis of the currently available data, it is not possible to demonstrate that the transmissible agent causing TSE is present in an infectious form in the blood and blood products (see P. Brown above). However, nor is it possible to state that it is absent owing, on the one hand, to the likelihood that levels in the blood would be very low, and on the other, to the very long incubation period of the disease after infection of the subject.

Given the sources of certain medicinal products, for example involving biological starting materials of human or animal origin potentially contaminated with a NCTA (blood, organs, bone, skin, plasma, placenta or cell cultures) or obtained via processes with the potential to contaminate the starting material (culture medium, growth factor), the pharmaceutical industry is currently evaluating the efficacy of the processes used to obtain or treat biological medicinal products, of equipment decontamination procedures used to eliminate NCTAs and finally, that of confinement procedures.

As the only known infectious agent (i.e. one capable of transmitting the disease) common to all NCTAs is the prion protein in the pathogenic form, PrPsc, the efficacy of these methods is evaluated with regard to the elimination or confinement of PrPsc.

The surprising resistance of PrPsc in fact precludes the use of the classic inactivation processes (for example treatment with TWEEN-TNBP solvent/detergent) known to reduce viral load in such blood products as cryoprecipitable plasma proteins (factor VIII, von Willebrand factor, etc.).

### PRIOR ART

Such methods used to evaluate and/or control the efficacy of the procedures by which biological medicinal products are obtained or processed, decontamination of equipment to eliminate PrPsc and confinement procedures must include:
a titration method of PrPsc in the biological substance before, during and after it is obtained, or processed or decontaminated
and an infecting inoculum, i.e. an infectious material allowing contamination of the biological product or the equipment to be decontaminated, with a known infectious PrPsc titer, the changes in which can be monitored.

As titration methods of PrPsc, the following methods are known.

Generally speaking, the prion infectious titration are performed by intracerebral inoculation of laboratory animals, with different dilutions of a product to be tested spiked with PrPsc, for example rodents which have already allowed the adaptation of infecting TSE strains from other animals, in particular the golden hamster, or transgenic mice expressing a PrP of the same species as PrPsc, the source of infectiousness. Depending on the number of animals infected in the different groups corresponding to the performed dilutions, the infectious titer can be calculated. However, this method is long, costly and difficult to develop on an industrial scale. The incubation period for ovine Scrapie in the golden hamster after intracerebral injection is 75 days.

Furthermore, known titration methods are available for the in vitro titration of TSE infectious agents. They consist in detecting PrPsc by Western-Blot (Ironside J. W. et al, J. of Thrombosis and Haemostasis, 1, 1479-1486, 2003) or by ELISA. These methods involve either preliminary digestion of the sample to be analysed using Proteinase K, or denaturation using chaotropic agents to identify the pathogenic protein (PrPsc) from the normal protein (PrP). Indeed, the antibodies detect the prion protein fragment which is proteinase K-resistant, called PrPres. Another titration method was recently developed on the basis on the use of specific PrPsc antibodies which do not recognise PrP. Similarly, in patent application No. WO 04/02179 the Applicant describes an in vitro titration method for a NCTA using stable transgenic cell lines supporting replication of the PrPsc corresponding to the aforementioned NCTA and leading to amplification of the quantity of PrPsc present in the biological substance to be tested, as well as the use of such a titration method as part of an in vitro evaluation and/or control method for processes used to obtain or treat a biological product potentially contaminated with a NCTA or as part of an in vitro evaluation and/or control method for processes used to decontaminate equipment. This titration method enables to calculate the infectious titer in PrPsc of a biological product that may be contaminated by a NCTA.

Examples A and B of this application are the reference Examples providing a reminder of the feasibility of the titration method described in patent application WO 04/02179 and of its use to evaluate the efficacy of a nanofiltration step vis-à-vis PrPsc elimination.

As regards the choice of the infecting inoculum suitable for such methods, the choice is extremely limited.

To date, a single infectious source has been used: an infecting inoculum consists of an infected brain homogenate.

In most cases, these homogenates are obtained from the brains of laboratory animals, particularly rodents, which have been infected by intracerebral inoculation with infected material.

Miekka et al. disclose the use of a brain homogenate obtained from hamsters infected with hamster 263K Scrapie protein as the infecting inoculum of a 25% albumin solution to evaluate the efficacy of gamma irradiation with regard to inactivation of hamster PrPsc while preserving the integrity of human albumin. It is also possible to use standard brain and spleen preparations obtained from subjects infected with new-variant Creutzfeld-Jacob Disease (vCJD) and available from the National Institute for Biological Standard and Controls (NIBSC). They consist in 10% tissue homogenates, which are aliquoted and stored. They present the advantage of being a human source of infectious material.

Stenland et al. (“Partitioning of human and sheep forms of the pathogenic prion protein during the purification of therapeutic proteins from human plasma”, TRANSFUSION, Vol 42, November 2002) compared a variety of brain homogenates obtained from several species and infected with different diseases with respect to the elimination of these diseases by a variety of purification steps of human plasma proteins
As infecting inocula, they used the following brain homogenates:
human, infected with new-variant Creutzfeld-Jacob disease (National CJD Surveillance Unit, Edinburgh, Scotland)
human, infected with sporadic Creutzfeld-Jacob disease (NIH Laboratory of CNS Studies, Bethesda, Md.)
human, infected with Gertsmann-Sträussler-Scheinker syndrome (New York State Institute for Basic Research in Developmental Disabilities, Staten Island, N.Y.)
sheep, infected with scrapie (Caine Veterinary Teaching Center, University of Idaho, Moscow, Id.)
hamster, infected with hamster-adapted 263K scrapie strain.

The homogenates were prepared by dispersing 1 g of tissue in 9 volumes of cold TBS (Tris Buffered Saline) followed by homogenisation.

They concluded that the brain homogenate of hamsters infected with hamster-adapted 263K scrapie was a suitable substitute for the human brain homogenates infected with the different NCTAs cited above.

Vey et al. (“Purity of Spiking Agent Affects Partitioning of Prions in Plasma Protein Purification”, Biologicals, 2002, 30, 187-196) compared different forms of infecting inocula of varying degrees of purity, i.e. brain homogenate, microsomal membrane fraction, “caveolae-like” domain (specialized membranous compartment), purified PrPsc (Bolton et al., Isolation and structural studies of the intact scrapie agent protein, Arch Biochem Biophys. 1987 Nov. 1; 258(2):579-90), with regard to their elimination, measured in PrPsc titer, by various plasma protein purification processes. The brain homogenates were prepared as 10% dispersions in cold TBS which were then homogenised on an Ultra Turrax. They concluded that the use of different infectious inocula with different properties should be preferred to simulate at best the elimination of the theoretical prion contaminant from the blood.

U.S. Pat. No. 6,020,537 discloses the use of transgenic mice expressing a PrP of the same species as the infectious source to manufacture reference samples for the detection of prions.

The company Haemosan LSS GmbH (Prof. Herwig Reichl) uses the SMB lymphocyte cell line spiked with scrapie strain 139β as experimental spikes to evaluate procedures by which biological products liable to be contaminated by a NCTA are obtained and their ability to eliminate the latter. This cell line is not transgenic and generates extremely low levels of infectiousness, which constitutes a major obstacle to the evaluation of steps in a manufacturing process by which products of biological origin are obtained.

### TECHNICAL PROBLEM

The use of a brain homogenate as an infectious inoculum engenders technical problems of incompatibility with the products to be tested or with elimination/inactivation methods.

Indeed, high lipid contents in the brain extract can induce the absorption of the biological product sought into the brain aggregates; similarly, the resulting insolubility can artificially increase the PrPsc reduction factor as the infected material then behaves differently from a soluble material, for example it may clog a filter or dry on the surface of the equipment to be decontaminated.

Diluted brain homogenates may, of course, be used. However, this is not compatible with a high infectious titer, particularly because the homogenate has already been diluted to 10% brain weight/volume of diluent.

Using a brain homogenate as an infectious inoculum also creates problems as regards:
the incubation time required for PrPsc replication
experimental animal costs
the need for qualified staff for laboratory animal handling
industrial scale-up
sample-to-sample reproducibility
and in parallel, the obvious ethical concerns related to the use and deliberate infection of animals.

In addition, infectious cerebral matter is hard to standardise. Indeed, it is difficult to reproduce the infectious dose in animals brains which results from intracerebral inoculation with infectious material. This is particularly the case when the infectious material used is itself a brain homogenate with an unknown infectious load.

Finally, the brain homogenate model in itself appears to be poorly suited to a potential infectious form in the blood and present in an entirely different environment.

Identification of a new source of infectiousness, with a comparable or even higher PrPsc infectious titer than that presented by infected brain homogenates, therefore appears necessary. This new source should also present advantages that will both dispense with the above mentioned problems related to the use of brain homogenates (solubility, standardisation, storage, reproducibility, simplified culture conditions) and allow it to be used as an infectious inoculum for evaluation and/or control methods applied to procedures by which biological products are obtained or processed, and for equipment decontamination procedures.

### SUMMARY OF THE INVENTION

The Applicant has identified, in an unusual manner, just such a synthetic, standardised, soluble, reproducible and easy to handle new source of infectiousness, consisting of a cell lysate or culture supernatant derived from stable transgenic cells expressing a prion protein PrP and supporting replication of the pathogenic form, PrPsc, of said PrP, whose infectious titer in PrPsc is greater than 50%, particularly 75% and in a preferred embodiment, 100% of the infectious titer of animal brain homogenates infected with PrPsc.

The cell lysate or culture supernatant described in the present invention, can be standardised and stabilised by methods known by the person skilled in the art.

This cell lysate or culture supernatant can be used as an infectious inoculum:
as part of an evaluation and/or control method for a procedure used to obtain or to process a biological product liable to be contaminated with a NCTA, notably procedures used to purify blood plasma products derivatives and in particular chromatographic methods or nanofiltration.
for a method used to evaluate and/or control the decontamination process for equipment liable to be contaminated with a NCTA.
for a method used to evaluate a compound inhibiting the infectiousness of a NCTA.

It would be possible to obtain infinite quantities of such an infectious inoculum with a high infectious titer, irrespective of the species infected with the NCTA, particularly humans, since the cell lines used allow the infectious material to be amplified in vitro.

The Applicant has identified the only way of obtaining infectious human material without having recourse to the use of patients' cerebral matter.

Similarly, the infectious material according to this invention dispenses with the need for using Scrapie or Bovine Spongiform Encephalitis-infected ovine or bovine cerebral tissues collections.

### DETAILED DESCRIPTION OF THE INVENTION

The invention therefore concerns a cell lysate or culture supernatant obtained from stable transgenic cells expressing a prion protein PrP and supporting replication of PrPsc, the pathogenic form of said PrP, whose infectious titer in PrPsc is greater than 50%, particularly 75% and preferably 100%, of the infectious titer of 10% animal brain homogenates infected with said PrPsc.

The use of such stable transgenic cells according to the invention presents many advantages, notably the possibility of ad infinitum production and immediate availability of the material as it is excreted in the culture supernatant.

Vilette et al. (PNAS, Mars 2001, 98(7), 4055-4059) demonstrated that transgenic, stable rabbit epithelial cells expressing ovine PrP (transgenic), are capable of replicating ovine PrPsc after infection with extracts containing the latter substance. The cell model they devised, the Rov9 line, inducibly expresses exogenous PrP, guaranteeing that it is maintained during the incubation period necessary for the accumulation of PrPsc in the cells exposed to an infectious matter. Such stable transgenic cells which support PrPsc replication are appropriate for use in the production of the infectious material according to this invention.

Archer et al. (Journal of Virology, January 2004, p. 482-490) created another type of stable cell line supporting the replication of a PrPsc. Their model consisted in murine glial cells, MovS cells, expressing ovine PrP and supporting the replication of ovine PrPsc. These cells showed themselves to be particularly suited to the production of infectious material according to this invention.

The 10% brain homogenate of animals infected with a PrPsc, whose infectious titer serves as a reference for the infectious titer in the same PrPsc of the cell lysate or culture supernatant according to this invention, is diluted to 10%, for example, in an isotonic buffer, for example in TBS or PBS. It is then homogenised in this diluent using, for example, an Ultra turrax.

The animal brains are said to be “infected with a PrPsc” when this infection is confirmed by histopathological analysis and/or after detection of the PrPres by immunohistochemical staining, and when said animals have developed the symptoms of the NCTA corresponding to said PrPsc. Particularly, the “brains of animals infected by a PrPsc” can be obtained from animals who have died from a NCTA-related disease and from moribund animals that have been euthanatized, if brain infection by PrPsc has been confirmed by histopathology and/or immunochemical staining.

The infectious titer can be determined by the titration method described in the international patent application WO 04/02179 filed by the Applicant.

The infectious titer may also be determined using methods which are well known by the person skilled in the art. For example, serial dilutions of the material to be titrated are injected intracerebrally to animals in which infection with the PrPsc to be titrated is possible, in this case at a rate of one dilution per group of animals. Depending on the incubation period of the disease in each group of animals and the number of animals affected, the infectious titer may be deduced using statistical methods known by the skilled person such as the Spearman-Kärber method.

Advantageously, the cell lysate or culture supernatant according to the invention can be standardised in infectious units per dose. In addition, it can be stabilised by freezing, drying, freeze-drying and atomisation, potentially in the presence of substances known by the person skilled in the art and which are intended to prevent loss of infectious titer related to the stabilisation method used.

Within the scope of this invention, the term “NCTA” refers to all NCTAs, including those responsible for both familial and sporadic CJD, Kuru disease, the CJD variant evidenced in young subjects, and also those causing ovine scrapie or bovine or feline spongiform encephalitis, in animals.

The term “prion protein” describes the signature protein of a NCTA, expressed in normal form, PrP, in the individuals of a given species liable to be infected by the NCTA in question, and expressed as the pathogen, PrPsc, in the individuals of this same species already infected with the NCTA. This protein is the infection carrier element present in all cases of NCTA infection, particularly in the brain, i.e. it is capable of inducing the symptoms of the NCTA after intracerebral inoculation in a healthy subject.

The cell lysate or culture supernatant according to the present invention has an infectious titer of 4, preferably 5, more preferably 6, and in a particularly preferred embodiment 7 log TCID₅₀/ml.

In particular, the infectious PrPsc present in the cell lysate or culture supernatant according to the invention is of ovine, bovine or human origin. Preferably, it represents the ovine prion protein in sheep scrapie or the human prion protein of new-variant Creutzfeld-Jacob disease.

One particular embodiment of the invention consists in comparing the infectious titer of the cell lysate or culture supernatant according to the invention, with an animal brain homogenate obtained from a given species and infected with a PrPsc of the same species, for example a sheeps brains homogenate from animals infected with the ovine PrPsc of scrapie, a hamsters brains homogenate from animals infected with the PrPsc of the hamster-adapted strain 263K, a bovine brain homogenate from animals infected with the bovine PrPsc of bovine spongiform encephalitis (BSE), or a human brains homogenate obtained from patients infected with the human PrPsc of new-variant Creutzfeld-Jacob disease (vCJD).

According to another embodiment of the invention, the infectious titer of the cell lysate or culture supernatant according to the invention, is compared with a brains homogenate obtained from transgenic animals expressing a PrP from a given species as a transgene and infected with a PrPsc from the same species, for example, a transgenic mice brains homogenate expressing ovine PrP and infected with ovine PrPsc. These transgenic animals can be “knockouts” (both alleles deleted) for the PrP gene of their own species.

In an advantageous manner, the cell lysate or culture supernatant according to the invention, is obtained from rabbit epithelial cells, particularly rabbit epithelial cells from the cell line Rov9 (Vilette et al.) or from murine glial cells, particularly murine glial cells from the cell line MovS6 (Archer et al.).

The present invention also concerns the use of infectious material according to the invention, as an infecting inoculum in a method for evaluating and/or controlling in vitro a procedure by which a biological product liable to be infected by an non-conventional transmissible agent (NCTA) is obtained or processed. This evaluation and/or control method is characterised by the following:
the infectious material according to the invention is inoculated into the biological product to simulate infection;
the biological product artificially infected with the infectious material according to the invention is titrated both before and after the said procedure
and the two PrPsc infectious titers obtained are compared.

By comparing the two measurements, the PrPsc degree of elimination or reduction factor is obtained

Thus, the implementation of the present method may be performed during a procedure used to obtain a biological product or within the scope of an elimination process of the NCTA once the biological product has been obtained.

Procedures used to obtain or process biological products are, in particular, processes by which blood products, such as plasma derivatives, are obtained or purified using, for example, chromatographic methods or nanofilatration, and in particular, the chromatographic methods described in patent EP 0 359 593 and in patent application WO 02092632.

The present invention also concerns the use of infectious material according to the invention as an infectious inoculum in an in vitro method for the evaluation and/or control of an equipment decontamination procedure. In this case, the equipment to be decontaminated by the decontamination method to be tested is artificially infected. To this end, the infectious material according to the invention, the infectious titer of which is known, is brought into contact with the equipment to be decontaminated which is then subjected to the decontamination procedure. Finally, the titer of a sample withdrawn from the decontaminated equipment after the decontamination cycle, is determined. This titer is compared to the initial infectious titer of the infectious material according to the invention to evaluate the efficacy of the decontamination procedure.

The equipment may consist of, for example, a purification system, more specifically a chromatography column.

The decontamination procedure may be, for example, the cleansing of a chromatographic column using sodium hydroxide.

The invention also concerns the use of the infectious material according to the invention as an infecting inoculum for a method to evaluate a compound inhibiting NCTA infectiousness. In this case, the NCTA infectious capacity of the infectious material according to the invention is tested with and without the compound liable to inhibit the infectiousness of said NCTA.

This invention also concerns the use of the infectious material according to the invention as an infecting inoculum for an evaluation and/or control method of an infectious material confinement procedure, particularly type P3 procedures and equipment.

### REFERENCE EXAMPLE A

### Titration of PrPsc PG127 Infectiousness by In Vitro Titration on Mov S6 Cells

In order to study the feasibility of an in vitro system intended to titrate the infectiousness related to NCTAs, MovS6 cells (Archer F. et al. 2004) were selected as cells supporting NCTA replication, and PG127 scrapie Strain adapted to Tg301 transgenic mice was used as source of infection (Vilotte J L et al., Journal of Virology, Vol. 75, n^{o}13, p. 5977-5984, 2001). The inoculum consisted of a 10% (weight/vol) mice brains homogenate infected at a rate of 100 mg/ml by PG127 scrapie Strain, i.e. with a 10% (weight/vol) PG127/98 sheeps brains homogenate (Veterinary Laboratory Agency, Addelstone, UK) (cf. Vilotte J L et al., Journal of Virology, Vol. 75, n^{o}13, p. 5977-5984, 2001, Material and Methods). The Mov S6 cells were cultivated in DMEM+HamF-12 medium supplemented with glutamine and foetal calf serum. The titration plates were prepared 24 hrs prior to inoculation. The cells were seeded at a rate of 40,000 cells per well on a 96-well plate (test No. 1), and 100,000 cells for a 24-well plate (tests 2 and 3). Ten-fold (tests 1 and 2) and 5-fold serial dilutions (test No. 3) of the mice brains homogenate were prepared with the culture medium. Five wells per culture plate were infected with each inoculum dilution. The cells were exposed to a given volume of inoculum (50 to 150 μl) for periods ranging from 12 hours to 4 days. Table 1 summarises the experimental inoculation and cells expansion conditions specific to each test. The inoculum was discarded and replaced with fresh culture medium, then the cells were maintained in culture for 72 hrs, until the first passage during which the culture plate format was changed to keep half (test No. 1), or all the infected cells (tests No. 2 and 3). The culture medium was subsequently changed once a week during the test, and the cells reseeded in a ratio of 1 to 10, allowing analysis of 90% of the cells at each passage. The cells harvested at each passage were frozen as cells pellet and stored at −80° C. until they were analysed using the Western-Blot method to detect PrPsc. Each cells pellet was treated with Benzonase (250 units) for 2 hours at 37° C. in a volume of 50 μl. The cell lysates were then treated with Proteinase K, denatured and then analysed using poly-acrylamide gel electrophoresis under denaturing conditions (SDS-PAGE). The proteins migrating in the gel were then transferred by electro-transfer onto a nitrocellulose membrane. The PrPsc present on the membranes was detected after incubation with 6H4 antibody (Prionics) then with a labelled secondary antibody (goat antibodies directed against mouse antibodies). The labelled membranes were revealed by chemiluminescence. A sample was deemed positive when the electrophoretic profile of the three forms of glycosylated PrPsc were visible on the autoradiogram. For each Western-Blot analysis, negative controls (uninfected MovS6 cells) were treated in parallel with the samples. At each cell passage, all the wells on the culture plates were tested. When all the cell culture replicates inoculated with a given inoculum dilution were found to be PrPsc-positive during two successive passages, these cultures were no longer tested during subsequent passages. The sample titer was calculated at the end of cell culturing using the Spearman Kärber method (Schmidt N J and Emmons R W, Diagnostic Procedures for Viral, Rickettsial and Chlamydial Infection 1989, 6th Edition).

TABLE 1
Experimental conditions applied for the in vitro
titration assays of NCTA-related infectiousness.

| Parameters | Test No. 1 | Test No. 2 | Test No. 3 |
|---|---|---|---|
| Culture format for | 96-well | 24-well plate | 24-well plate |
| inoculation | microplate | | |
| Cell density for | 40 000 | 100 000 | 100 000 |
| seeding | | | |
| (cells/well) | | | |
| Volume of inoculum | 50 | 1 100 | 150 |
| (μl) | | (100 μl | |
| | | diluted in 1 | |
| | | ml of fresh | |
| | | medium) | |
| Initial contact | 12 | 96 | 24 |
| time (hours) | | | |
| Addition of fresh | 0.2 | 1 | 1 |
| medium (ml) | | (inoculum | |
| | | discarded) | |
| Secondary | 48 with diluted | 24 | 72 |
| incubation (hours) | inoculum, then | | |
| | 48 with fresh | | |
| | medium. | | |
| Percentage of cells | 50% | 100% | 100% |
| reseeded at first | | | |
| passage | | | |
| (change in 6-well | | | |
| format) | | | |

### Results

### Test No. 1

10⁻¹ to 10⁻⁶ dilutions of mice brains homogenates infected with PG127 scrapie Strain were tested. At passage No. 3, none of the cell wells presented PrPsc. At passage No. 4, 100% of the wells that had been inoculated with dilutions 10⁻¹ to 10⁻³ of the inoculum were positive for PrPsc, and 75% of the cultures infected with the 10⁻⁴ dilution were positive. At passage No. 5, 25% of the wells inoculated with dilution 10⁻⁵ were positive, giving a titer of 50% infectious dose (TCID₅₀) of 6.05 log per ml as per the Spearman Karber method. The titer of the stock did not increase during the subsequent cell passages.

### Test No. 2

Table 2 summarises the results obtained during test No. 2, which once again show the changes in the percentage of positive results in each culture replicate infected with each of the inoculum dilutions tested (from 10⁻³ to 10⁻⁷). It can be observed that the time to onset of PrPsc in the cultures is inversely proportional to the infectious dose with which the cultures were inoculated. Thus, the cultures receiving the 10⁻³ dilution of the inoculum presented PrPsc at passage No. 2, while an additional passage was required for 100% of the cultures inoculated with the 10⁻⁴ to be positive. The inoculum titer calculated for this experiment was 5.5 TCID₅₀/ml.

TABLE 2
Results of titration test No. 2.

| | Number of cultures infected as a | | | | | | |
|---|---|---|---|---|---|---|---|
| | function of dilution inoculated | | | | | | |
| | (wells infected/wells inoculated) | | | | | | Titer |
| Passage | | | | | | Neg. | (log |
| No. | 10⁻³ | 10⁻⁴ | 10⁻⁵ | 10⁻⁶ | 10⁻⁷ | control | TCID₅₀/ml) |
| 1 | NT | NT | NT | NT | NT | NT | |
| 2 | 5/5 | 1/5 | 0/5 | 0/5 | 0/5 | 0/5 | 4.7 |
| 3 | 5/5 | 5/5 | 0/5 | 0/5 | 0/5 | 0/5 | 5.5 |
| 4 | NT^{a} | 5/5 | 0/5 | 0/5 | 0/5 | 0/5 | 5.5 |
| 5 | NT^{a} | NT^{a} | 0/5 | 0/5 | 0/5 | 0/5 | 5.5 |
| 6 | NT^{a} | NT^{a} | 0/5 | 0/5 | 0/5 | 0/5 | 5.5 |

NT: Not tested as 100% of the cells were seeded
NT^{a}: Not tested, as 100% positive on two preceding passages.

### Test No. 3

Table 3 summarises the results obtained during test No. 3. For the purposes of this test, the inoculum dilution rate was reduced to 1/5, and the dilutions tested were 10⁻⁴ to 10^{-6.8}. The maximum titers were obtained at passage No. 6, and the titer calculated was 6.43 TCID₅₀/ml.

TABLE 3
Results of test No. 3.

| | Number of cultures infected | | | | | | |
|---|---|---|---|---|---|---|---|
| | depending on dilution inoculated | | | | | | |
| | (infected wells/inoculated wells) | | | | | | Titer |
| Passage | | | | | | Neg. | (log |
| No. | 10⁻⁴ | 10^{-4.7} | 10^{-5.4} | 10^{-6.1} | 10^{-6.8} | control | TCID₅₀/ml) |
| 1 | NT | NT | NT | NT | NT | NT | |
| 2 | 1/5 | 0/5 | 0/5 | 0/5 | 0/5 | 0/5 | 4.6 |
| 3 | 5/5 | 3/5 | 0/5 | 0/5 | 0/5 | 0/5 | 5.6 |
| 4 | 5/5 | 5/5 | 3/5 | 0/5 | 0/5 | 0/5 | 6.3 |
| 5 | NT^{a} | 5/5 | 3/5 | 0/5 | 0/5 | 0/5 | 6.3 |
| 6 | NT^{a} | NT^{a} | 4/5 | 0/5 | 0/5 | 0/5 | 6.4 |
| 7 | NT^{a} | NT^{a} | 4/5 | 0/5 | 0/5 | 0/5 | 6.4 |

NT: Not tested as 100% of the cells were seeded
NT^{a}: Not tested, as 100% positive on two preceding passages.

The results of the three tests carried out demonstrate the feasibility of in vitro titration in TCID₅₀ of NCTA-related infectiousness.

### REFERENCE EXAMPLE B

### Calculation of a Reduction Factor Associated with a Purification Step for a Biological Medicinal Product Potentially Infected by a NCTA

For this test, a nanofiltration step was chosen as an example of one of the steps in a manufacturing process by which a biological product may be obtained. A PLANOVA 15 N (ASAHI KASEI) filter with a pore size of 15 nm and a surface area of 0.01 m² was selected. The substance to be filtered was 0.2 g/l human albumin in 0.01M trisodium citrate dihydrate buffer, 0.12M glycine, 0.016M L-lysine (monochloride), 0.001M calcium dihydrate chloride, 0.17M sodium chloride, with an osmolarity of 490-510 mosmol/kg and a pH of 6.90-7.10. Nanofiltration was performed at 25° C. under a pressure of 500±100 mbar. The filter was equilibrated with 40 ml of the buffer solution used to dilute the human albumin before nanofiltration of the product. Thirty (30) ml of the product to be filtered, spiked with 4% of the 10% mice brains homogenate (weight/volume) infected with PG127 Scrapie strain was filtered, then 10 ml of the buffer used to equilibrate the filter was filtered to flush the product through the filter. Two nanofiltration tests were carried out to ensure that this step was reproducible. The experimentally spiked product to be filtered was prepared in a quantity sufficient to withdraw an aliquot fraction to quantify the infectiousness present within the product before nanofiltration. A total volume of 40 ml of filtrate was recovered after nanofiltration, and titrated to determine the amount of infectiousness in the product after nanofiltration.

The starting spiked material and the filtered product were diluted at 1/3 in culture medium before being stored at −80° C. waiting for titration. The samples were titrated according to the experimental conditions for Test No. 3 described in the first example. For the filtered samples whose residual infectious titer was expected to be nil or very low, the lowest dilution of the non cytotoxic sample was inoculated into ten replicates, and the following dilutions into five replicates as described above.

Table 4 summarises the results of titration of the four samples generated by this nanofiltration test.

The titer of the starting spiked material was 4.67 log TCID₅₀/ml for tests 1 and 2. No residual infectiousness was detected in the filtrate samples.

Using the Poisson law, the infectious load of these samples was calculated to be 0.28 log TCID₅₀/ml from the volume of the lowest dilution of the inoculated sample. The total load present in the samples was calculated by multiplying their load by their respective volumes, and the reduction factor was calculated by dividing the load measured in the starting material, or the initial sample, before filtration by the load measured in the filtrates. The clearance factors were calculated from the calculated quantity of infectiousness added in the experimental spike. The difference between the clearance factor and the reduction factor, when it is above one log, allows identification of potential interference between the starting product and the capacity of the titration system to detect infectiousness in this product. Table 5 summarises these calculations. The reduction factors associated with the elimination of infectiousness present in the brain homogenate used to spike the starting material were ≧4.24 and ≧4.22 log for tests 1 and 2, respectively. Calculation of the clearance factors did not show any interference of the starting material.

TABLE 4
titration results for samples generated during
evaluation of a nanofiltration step.

| | | Number of cultures | |
|---|---|---|---|
| | Last positive | infected/number of | Titer |
| Sample | dilutions | cultures inoculated | (log TCID₅₀/ml) |
| Initial sample | 1/9375 | 5/5 | 5.15 |
| test 1 | 1/46875 | 0/5 | |
| Filtrate | Pure | 0/15 | <0.76* |
| test 1 | | | |
| Initial sample | 1/9375 | 3/5 | 5.15 |
| test 2 | 1/46875 | 2/5 | |
| Filtrate | Pure | 0/15 | <0.76* |
| test 2 | | | |

*no infectiousness detected: limit of detection calculated by Poisson law

TABLE 5
Calculation of reduction factors associated with the 15
nm nanofiltration step evaluated by in vitro titration

| | Titer | | Total load | Clearance | Reduction |
|---|---|---|---|---|---|
| | (log | Volume | (log | factor | factor |
| Sample | TCID₅₀/ml) | (ml) | TCID₅₀) | (log)¹ | (log)² |
| starting | 6.43 | 1.21³ | 6.51 | | |
| stock | | | | | |
| test 1 | | | | | |
| Initial | 4.67 | 30 | 6.63 | | |
| sample | | | | | |
| test 1 | | | | | |
| Filtrate | <0.76⁴ | 41.9 | <2.10 | ≧4.11 | ≧4.23 |
| test 1 | | | | | |
| starting | 6.43 | 1.28³ | 6.54 | | |
| stock | | | | | |
| test 2 | | | | | |
| Initial | 5.15 | 30 | 6.63 | | |
| sample | | | | | |
| test 2 | | | | | |
| Filtrate | <0.76⁴ | 44.3 | <2.13 | ≧4.12 | ≧4.20 |
| test 2 | | | | | |

¹calculated from the total load in the experimental spike.
²calculated from the initial load measured in the starting material.
³volume taking into account withdrawal of the aliquot fraction intended for titration of the initial sample.
⁴no detectable infectiousness: limit of detection as calculated by Poisson law

### EXAMPLE 1

### Titration of MovS6 Cell Lysate Infected with PG127 Strain Using the Titration Method Described in Example 1

The titration method described in Example 1 with the experimental conditions of test 3 was used, the only difference being the infecting inoculum.

The infecting inoculum used was a Mov S6 infected cells lysate as described in Example 1 with a 10% mice brains homogenate (weight/vol) infected at a rate of 100 mg/ml with PG127 scrapie strain.

Serial 10-fold dilutions, ranging from 10⁻⁴ to 10⁻⁸, were used to infect 5 wells per culture plate and per dilution.

The results are presented in Table 6.

TABLE 6
Titration of a lysate of MovS6 cells infected with strain PG127

| | Number of cultures infected | | | | | | |
|---|---|---|---|---|---|---|---|
| | with the inoculated dilution | | | | | | |
| | (infected wells/inoculated wells) | | | | | | Titer |
| Passage | | | | | | Neg. | (log |
| No. | 10⁻⁴ | 10⁻⁵ | 10⁻⁶ | 10⁻⁷ | 10⁻⁸ | control | TCID₅₀/ml) |
| 1 | NT | NT | NT | NT | NT | NT | |
| 2 | ND | ND | 0/5 | 0/5 | 0/5 | 0/5 | ND |
| 3 | 5/5 | 3/5 | 0/5 | 0/5 | 0/5 | 0/5 | 5.92 |
| 4 | 5/5 | 5/5 | 2/5 | 1/5 | 0/5 | 0/5 | 6.92 |
| 5 | NT^{a} | NT^{a} | 4/5 | 3/5 | 0/5 | 0/5 | 7.72 |
| 6 | NT^{a} | NT^{a} | 5/5 | 3/5 | 0/5 | 0/5 | 7.92 |
| 7 | NT^{a} | NT^{a} | 5/5 | 3/5 | 0/5 | 0/5 | 7.92 |

ND: not determined owing to technical problem
NT^{a}: Not tested because 100% positive for previous two passages
NT: Not tested because 100% of the cells were reseeded

### EXAMPLE 2

### Titration of a Culture Supernatant of MovS6 Cells Infected with Strain PG127 Using the Titration Method as Described in Example 1

The titration method described in Example 1 with the experimental conditions of test 3 was used, the only difference being in the infecting inoculum.

The infecting inoculum used was a culture supernatant of Mov S6 cells infected as described in Example 1 with a 10% mice brains homogenate (weight/vol) infected at a rate of 100 mg/ml with PG127 scrapie strain.

Serial 3-fold dilutions from 1/3 to 1/2187, were used to infect 5 wells per culture plate and per dilution. The results are presented in Table 7.

TABLE 7
Titration of a culture supernatant of
MovS6 cells infected with strain PG127

| | Number of cultures infected | | | | | | |
|---|---|---|---|---|---|---|---|
| | with the inoculated dilution | | | | | | |
| | (infected wells/inoculated wells) | | | | | | Titer |
| Passage | | | | | | Neg. | (log |
| No. | 1/27 | 1/81 | 1/243 | 1/729 | 1/2187 | control | TCID₅₀/ml) |
| 3¹ | 5/5 | 2/5 | 0/5 | 0/5 | 0/5 | 0/5 | 2.68 |
| 4 | 5/5 | 4/5 | 2/5 | 2/5 | 0/5 | 0/5 | 3.35 |
| 5 | NT | 5/5 | 4/5 | 2/5 | 0/5 | 0/5 | 3.54 |
| 6 | NT | 5/5 | 5/5 | 2/5 | 0/5 | 0/5 | 3.64 |
| 7 | NT | NT | 5/5 | 3/5 | 0/5 | 0/5 | 3.73 |

¹at the 3rd passage, all the replicates of dilutions ⅓, 1/9 and 1/27 were positive. The preceding passages were not tested
NT: Not tested because 100% positive for previous two passages.

### EXAMPLE 3

### Comparison of PrPsc and Infectiousness Titers

The infectiousness titers are summarised in Table 8 from Tables 3, 6 and 7.

Moreover, the Western-Blot method as per Example 1 was used to quantify PrPsc in the starting inocula: 10% (weight/vol) mice brains homogenate infected with 100 mg/ml PG127 scrapie strain, cell lysate of Mov S6 cells infected as described in Example 1 with a 10% (weight/vol) mice brains homogenate infected with 100 mg/ml PG127 scrapie strain and a cell culture supernatant of Mov S6 cells infected as described in Example 1 with a 10% (weight/vol) mice brains homogenate infected with 100 mg/ml PG127 scrapie strain. For the latter, it was necessary to concentrate PrPsc by ultracentrifugation before it could be detected.

Each inoculum was serially diluted in SDS-PAGE loading buffer. The first dilution of the inoculum no longer presenting the signals specific to PrPsc was considered to contain one Western-Blot unit. The inoculum titer in PrPsc is calculated to be the inverse of the limit dilution, taking into account the volume of each sample of each dilution loaded in the gel.

TABLE 8

| | Western-Blot | Infectious titer |
|---|---|---|
| | PrPsc titer | by in vitro |
| | (log unit | titration |
| Inoculum | WB/ml) | (TCID₅₀/ml) |
| Homogenate of mice | 5.86 | 6.43 |
| brains infected with | | |
| PG127 | | |
| Infected Mov S6 | 5.86 | 7.92 |
| cells lysate | | |
| Infected MovS6 cells | 2.16 | 3.73 |
| culture supernatant | | |

1. Cell lysate or culture supernatant obtained from stable transgenic cells expressing a prion protein PrP and supporting replication of the pathogenic form, PrPsc, of said PrP, whose infectious titer in PrPsc is greater than 50% of the infectious titer of a 10% homogenate of animals brains infected with said PrPsc.

2. Cell lysate or culture supernatant obtained from stable transgenic cells expressing a prion protein PrP and supporting replication of the pathogenic form, PrPsc, of said PrP, whose infectious titer in PrPsc is greater than 75% of the infectious titer of a 10% homogenate of animals brains infected with said PrPsc.

3. Cell lysate or culture supernatant obtained from stable transgenic cells expressing a prion protein PrP and supporting replication of the pathogenic form, PrPsc, of said PrP, whose infectious titer in PrPsc is greater than the infectious titer of a 10% homogenate of animals brains infected with said PrPsc.

4. Cell lysate or culture supernatant according to claim 1, characterised by an infectious titer of ≧4 log TCID₅₀/ml.

5. Cell lysate or culture supernatant according to claim 1, characterised by an infectious titer of ≧5 log TCID₅₀/ml.

6. Cell lysate or culture supernatant according to claim 1, characterised by an infectious titer of ≧6 log TCID₅₀/ml.

7. Cell lysate or culture supernatant according to claim 1, characterised by an infectious titer of ≧7 log TCID₅₀/ml.

8. Cell lysate or culture supernatant according to claim 1, characterised in that the prion protein PrP expressed by the stable transgenic cells supporting the replication of the pathogenic form PrPsc is of ovine, bovine or human origin.

9. Cell lysate or culture supernatant according to claim 8, characterised in that the prion protein PrP is the ovine prion protein of sheep Scrapie.

10. Cell lysate or culture supernatant according to claim 8, characterised in that the prion protein PrP is the human prion protein of new-variant Creutzfeld-Jacob disease.

11. Cell lysate or culture supernatant according to claim 1, characterised by the fact that the infected animals brains homogenate is obtained from the brains of animals of a given species infected with a PrPsc of the same species.

12. Cell lysate or culture supernatant according to claim 11, characterised in that the infected animals brains homogenate is obtained from the brains of sheeps infected with sheep scrapie ovine PrPsc.

13. Cell lysate or culture supernatant according to claim 11, characterised in that the infected animals brains homogenate is obtained from the brains of hamsters infected with hamster adapted PrPsc strain 263K.

14. Cell lysate or culture supernatant according to claim 11, characterised in that the infected animals brains homogenate is obtained from the brains of cattle infected with bovine spongiform encephalitis (BSE) bovine PrPsc.

15. Cell lysate or culture supernatant according to claim 11, characterised in that the infected animals brains homogenate is obtained from the brains of human patients infected with the human PrPsc of new-variant Creutzfeld-Jacob disease (vCJD).

16. Cell lysate or culture supernatant according to claim 1, characterised in that the infected animals brains homogenate is obtained from the brains of transgenic animals expressing a PrP of a given species as a transgene and infected with a PrPsc of the same species.

17. Cell lysate or culture supernatant according to claim 16, characterised in that the infected animals brains homogenate is obtained from the brains of transgenic mice expressing ovine PrP and infected with ovine PrPsc.

18. Cell lysate or culture supernatant according to claim 1, obtained from stable transgenic epithelial rabbit cells.

19. Cell lysate or culture supernatant according to claim 18, obtained from stable transgenic epithelial rabbit cell line Rov9.

20. Cell lysate or culture supernatant according to claim 1, obtained from stable transgenic murine glial cells.

21. Cell lysate or culture supernatant according to claim 20, obtained from stable transgenic murine glial cell line MovS6.

22. Use of a lysate or culture supernatant according to claim 1, as an infecting inoculum for an evaluation and/or control method applied to a procedure used to obtain or process a biological product with the potential to be contaminated by a NCTA.

23. Use of a lysate or culture supernatant according to claim 22, as an infecting inoculum for an evaluation and/or control method applied to a procedure used to purify blood plasma derivative products.

24. Use of a lysate or culture supernatant according to claim 23, characterised in that the purification process implies chromatographies or nanofiltration.

25. Use of a lysate or culture supernatant according to claim 1, as an infecting inoculum for an evaluation and/or control method applied to a procedure used to decontaminate equipment potentially contaminated with a NCTA.

26. Use of a lysate or culture supernatant according to claim 1, as an infecting inoculum for a method evaluating a compound inhibiting the infectiousness of a NCTA.

27. Use of a lysate or culture supernatant according to claim 1, as an infecting inoculum for an evaluation and/or control method for an infectious material confinement procedure.
